(19) United States
(12) Patent Application Publication
(10) Pub. No.: US 2017/0007630 A1
(43) Pub. Date: Jan. 12, 2017

(54) COMPOSITION AND METHOD OF DELIVERY OF L-ARABINOSE AND SELECT COMPOUNDS

(71) Applicant: Jimmy Lu, Alpine, UT (US)

(72) Inventor: Jimmy Lu, Alpine, UT (US)

(21) Appl. No.: 15/000,984

(22) Filed: Jan. 19, 2016

Related U.S. Application Data
(62) Division of application No. 13/666,383, filed on Nov. 1, 2012, now abandoned.
(60) Provisional application No. 61/553,950, filed on Nov. 1, 2011.

(51) Int. Cl. A61K 31/7004; A61K 36/07; A61K 36/24; A61K 36/16; A23L 33/10; A61K 31/715; A61K 31/10; A61K 31/404; A61K 31/7028; A23L 29/30; A61K 31/7034; A61K 45/06

(52) U.S. Cl. CPC A61K 31/7004; A61K 31/7034; A61K 36/07; A61K 36/24; A61K 36/16; A61K 45/06; A61K 31/715; A61K 31/10; A61K 31/404; A61K 31/7028; A23L 29/30; A23L 33/10; A23V 2002/00

(57) ABSTRACT

The disclosure relates to dietary supplement compositions and methods of use thereof comprising L-arabinose and one or more select compounds, which may include one or more select phytochemical compounds. The claimed compositions are useful in dietary supplements to facilitate mitigating elevated blood sugar levels and managing a user's weight as well as enhancing performance of digestive system to prevent inflammation and infection.

### CROSS-REFERENCE TO RELATED APPLICATIONS

This application is a division of co-pending U.S. patent application Ser. No. 13/666,383 filed Nov. 1, 2012, entitled “A COMPOSITION AND METHOD OF DELIVERY OF L-ARABINOSE AND SELECT COMPOUNDS,” which claimed the benefit of U.S. Provisional Application No. 61/553,950, filed Nov. 1, 2011, which are hereby incorporated by reference herein in their entireties, including but not limited to those portions that specifically appear hereinafter, the incorporation by reference being made with the following exception: In the event that any portion of the above-referenced applications is inconsistent with this application, this application supersedes said portion of said above-referenced applications.

### STATEMENT REGARDING FEDERALLY SPONSORED RESEARCH OR DEVELOPMENT

Not Applicable.

### BACKGROUND

The disclosure relates generally to dietary supplements comprising L-arabinose and one or more select compounds and methods of use and method for supplementing the diet for improving health, including using the dietary supplement to manage blood glucose levels. The disclosure relates to a dietary supplement for correcting a dietary deficiency, including a diet having excess levels of sucrose. The composition of the disclosure combines a nutritionally effective amount of Arabinose, or L-arabinose, and one or more select compounds.

Sucrase in the human body breaks down sucrose, a disaccharide, into fructose and glucose, two monosaccharides in equal amount. These simple sugars (simple carbohydrates) are readily absorbed and used by the human body (and by other mammalian bodies) to provide energy. Without the sucrase, a mammal body, such as human body, does not readily absorb the disaccharides. Unfortunately, humans and other mammals may consume too much sucrose and so the body has to digest and deal with the excess broken down sucrose compounds.

In addition, consuming large quantities of sucrose causes the body to rely on fructose and glucose available from the sucrose to provide quick, readily available energy. Sucrose consumed by a mammal is converted to fructose and glucose through hydrolysis by the enzyme sucrase. If the body does not use up or fully digest the monosaccharides (i.e. fructose and glucose) generated through hydrolysis of sucrose then the body must manage them.

One way the body manages excess sucrose monosaccharides (i.e. fructose and glucose) is by storing the sugars as fat especially visceral fat which produces LDL and triglycerides. This can lead to increased fat production, weight gain, heart diseases and other health problems. Some bodies are unable to effectively manage excess monosaccharides (i.e. fructose and glucose), which can lead to elevated blood sugar levels. These elevated blood sugar levels can be particularly problematic to those who have diabetes.

Sucrase is an enzyme that catalyzes the hydrolysis (breakdown) of sucrose into fructose and glucose. Sucrose is a disaccharide compound formed from two monosaccharides molecules. Sucrase is the catalyst for the chemical separation of sucrose into fructose and glucose in the presence of water (H2O). Sucrase is secreted by the villi of the intestine of the mammal. Diets that are high in sucrose tend to lead to adverse health conditions, such as obesity, type 2 diabetes, cardiovascular diseases, mood swings, and the like. Furthermore, mammals with a diet high in sucrose tend to less effectively use other macronutrients, and instead tend to rely more on the sucrose intake to provide a ready source of quick energy.

It is noteworthy that no composition, known to the inventor, provides a compound that can assist the mammal body in handling excess sucrose along with excess monosaccharide molecules of fructose and glucose in the digestive tract. There is a long felt, but currently unmet, need for handling excess sucrose along with excess monosaccharide molecules of fructose and glucose in the digestive tract.

Known products are characterized by several disadvantages that may be addressed by the disclosure. The features and advantages of the disclosure will be set forth in the description which follows, and in part will be apparent from the description, or may be learned by the practice of the disclosure without undue experimentation. The features and advantages of the disclosure may be realized and obtained by means of the combinations particularly pointed out in the appended claims.

### SUMMARY OF THE DISCLOSURE

Compositions in accordance with one or more of the embodiments of the disclosure reduce the catabolism of sucrose and add a variety of other health benefits. Compositions in accordance with one or more of the embodiments of the disclosure remove fructose and glucose as energy sources in the diet by inhibiting the hydrolysis of sucrose. Instead of entering the circulative system to be absorbed by the body, the sucrose either aides the digestive system by feeding the probiotics in the large intestine area or passes through the digestive system. Select compounds from plants and fungi may be utilized in the compositions of the disclosure to aid the body with anti-inflammatory, anti-infection and immunoregulative effects to prevent the sucrose that is being blocked by L-arabinose. Unblocked sucrose will be hydrolyzed or broken down into fructose and glucose, which cause unexpected health issues, such as inflammation, infection or immune system unbalances. Select compounds from plants and fungi may be used to work with L-arabinose and the blocked sucrose to feed probiotics to promote colon health and to block absorption of other non-sucrose sugars, such as starch, and reabsorption of glucose in the blood by the co-transporters, such as SGLT-1 and SGLT-2, and other health benefits.

As a consequence, the body shifts from relying on a ready energy source of fructose and glucose to other macronutrients, such as fatty acids (fat), amino acids (protein), and other carbohydrates, other than sucrose. These macronutrients, fatty acids (fat), amino acids (protein), and other carbohydrates, other than sucrose, are referred to herein as the non-sucrose macronutrients. The inventor believes that the body shifts from relying on sucrose as an energy source to the non-sucrose macronutrients because the body still has a minimum, or average, number of calories that are needed each day to sustain itself (typically the average number of calories needed each day by humans is about 2,000 calories). Provided other dietary sources of energy are not consumed, the inhibiting of sucrose catabolism results in a shift to other energy sources to meet this minimum or average daily requirement. The body shifts to using other carbohydrates and proteins or even to breaking down stored fats to generate the same level of energy that would have come from the broken down sucrose.

The purpose to the composition and method of the disclosure is to make L-Arabinose more readily available to larger number of users to inhibit sucrose and to block sucrose intake, which may be harmful to human health, by utilizing the anti-allergic inflammation effect of PSK, a selected extract from a mushroom or other phytochemicals that possesses same kind of anti-allergic inflammation property. Sucrose has been proven to be a cause, if not the main cause, of metabolic syndrome, central nervous system syndrome and many other chronical diseases as well as accelerated aging through its contribution to the glycation process and the formation of AGEs, Advanced Glycation End-Products. Thus, it is desirable to actively block sucrose, which is added sugar and a potentially addictive sweetener existing in almost all food items available today. L-Arabinose inhibits sucrase so sucrose will not be processed and blocked from entering into the circulatory system. However, just like lactose to some people, sucrose becomes an allergen when it enters the large intestine to cause discomforts, such as loose bowl or even diarrhea. In one study conducted in China about 40% of the participants experienced that condition. That is one of the reasons that has caused L-Arabinose to not be successfully marketed.

Applicant has found that polysaccharides from mushrooms in the Agaricomycetes class may offer unexpectedly good results. Among them, polysaccharide extracts known as PSK, PSP and PST from mushrooms like Trametes versicolor and Coriolus versicolor, have been found to be effective in controlling the above mentioned allergic inflammation. With a range of ratios between 5-10%, a mix of L-arabinose and selected polysaccharides made possible for users to enjoy the sugar blocking effect of L-arabinose without suffering from the only known side effect.

With the addition of the polysaccharides in the formula, the work of dietary fibers including blocked sucrose, the ability for L-arabinose to serve as prebiotics to feed probiotics in the large intestine is enhanced. It not only prevented disorder to cause early discharge of the residue of food, but also to help probiotics to benefit from the dietary fibers to establish and maintain a healthy condition of the large intestine area where as much as 80% of immune activities take place.

The third active ingredient may be Konjac Glucomannan, or Konjac mannan, an extract from the root of Amorphophalms konjac, which serves three purposes: (1) to act as an binding agent to bind L-arabinose and polysaccharides to make the resulting composition or product easy carrying and easy dosing; (2) to act as an appetite suppressant to help weight management; and (3) to act as another dietary fiber for other health benefits.

### DETAILED DESCRIPTION

For the purposes of promoting an understanding of the principles in accordance with the disclosure, reference will now be made to certain embodiments and specific language will be used to describe the same. It will nevertheless be understood that no limitation of the scope of the disclosure is thereby intended. Any alterations and further modifications of the inventive features discussed and disclosed herein, and any additional applications of the principles of the disclosure, which would normally occur to one skilled in the relevant art and having possession of this disclosure, are to be considered within the scope of the disclosure claimed.

Before the compositions and methods for supplementing the diet for improving health and preventing disease are disclosed and described, it is to be understood that this disclosure is not limited to the particular compositions, configurations, process steps, and ingredients disclosed herein as such compositions, configurations, process steps, and ingredients may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise. Thus, for example, reference to a dietary supplement comprising or containing “a phytochemical” includes a mixture of one or more of such phytochemicals.

As used herein, the terms “comprising,” “including,” “containing,” “characterized by,” and grammatical equivalents thereof are inclusive or open-ended terms that do not exclude additional, unrecited ingredients, elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified ingredients, materials or steps and those that do not materially affect the basic and novel characteristic or characteristics of the claimed disclosure.

As used herein, “effective amount” means an amount of an ingredient or component of the dietary supplement that is nontoxic, but sufficient to provide the desired effect and performance at a reasonable benefit/risk ratio attending any dietary supplement. An effective amount of L-arabinose is an amount sufficient to block the catabolism of sucrose at a level sufficient to reduce the incidence of health issues, such as such as inflammation, infection or immune system unbalances caused by the presence of excess sucrose along with excess monosaccharide molecules of fructose and glucose.

In an embodiment, a composition in accordance with the disclosure provides a nutritional supplement that operates to inhibit the natural process of the enzyme sucrase of breaking down sucrose into the monosaccharide molecules of fructose and glucose. The dietary supplement is a composition that may comprise L-arabinose and one or more select compounds. In certain embodiments, the select compounds may be or contain phytochemical compounds, herbs, and/or extracts that may be selected from the group comprising one or more of phlorizin, coriolus versicolor, fomes yucatanensis, white kidney bean, and gymnema sylvestre extract. In other embodiments, the select compounds may be or contain phytochemical compounds, herbs, and/or extracts selected from the group comprising one or more of a terpene (isoprenoid), a polyphenolic compound, and a saponin.

In an embodiment, a composition in accordance with the disclosure may comprise a biologically effective composition comprising a range of about twenty parts to about one part of L-arabinose and one part of one or more of the select compounds. In an embodiment, a composition in accordance with the disclosure may comprise a biologically effective composition comprising ten parts L-arabinose and one part of one or more of the select compounds. In an embodiment, a composition in accordance with the disclosure may comprise a biologically effective composition comprising eight parts L-arabinose and one part of one or more of the select compounds. In an embodiment, a composition in accordance with the disclosure may comprise a biologically effective composition comprising five parts L-arabinose and one part of one or more of the select compounds. In an embodiment, a composition in accordance with the disclosure may comprise a biologically effective composition comprising three parts L-arabinose and one part of one or more of the select compounds. In an embodiment, a composition in accordance with the disclosure may comprise a biologically effective composition comprising one part L-arabinose and one part of one or more of the select compounds. Further, L-arabinose may be present in the composition in an amount that is greater than about fifty percent by weight, or in an amount that may be between a range of about fifty-five percent to about ninety-nine percent by weight, or in an amount that may be between a range of about eighty percent to about ninety-five percent by weight, or about eighty-three percent to about ninety-five percent by weight.

A phytochemical is a chemical compound that occurs naturally in plants and fungi. As used herein, the term “phytochemical” refers to plant synthesized molecules, found in food, or plant tissue in a complex organic matrix, or in fungi, which are minimally altered by processing from how they occur in nature. Phytochemicals can be extracted from a variety of plants and fungi, such as herbs, fruits, vegetables and mushrooms. See en.wikipedia.org/wiki/Phytochemical (last visited Oct. 25, 2012) for more information on phytochemicals. In certain embodiments, the one or more select compounds are derived from phytochemicals, plant extracts, and fungi extracts. In certain embodiments, the dietary supplement may be a composition comprising L-arabinose and one or more phytochemicals, including, for example, polysaccharides. It may be advantageous in certain embodiments of the disclosure, to use the one or more select compounds that come from plants or fungi known to have medicinal and herbal benefits such as, for example, the organic or carbon based phytochemicals. In addition, it should be noted that L-arabinose can be combined with a fungus and/or fungus extracts to accomplish similar benefits to those described herein in relation to a combination of L-arabinose and the one or more select compounds. Representative examples of phytochemicals that can be used with L-arabinose include, but are not limited to: phenolic compounds, terpenes (isoprenoids), betalains, organosulfides, indoles, glucosinolates, sulporane, naringin, hesperidin, narirutin, quercetin, B-carotene, lutein, lycopene, isoflavones, protein inhibitors, other organic acids, and the like.

The phytochemical compound may be present in the composition in an amount that is less than fifty percent by weight, or in an amount that may be within a range of about forty percent to about two percent by weight, or in an amount that may be within a range of about twenty percent to about five percent by weight.

Phenolic compounds include, but are not limited to, monophenols, flavonoids (polyphenols), phenolic acids, hydroxycinnamic acids, lignans (phytoestrogens), tyrosol esters, stilbenoids, punicalagins, and the like.

Terpenes include, but are not limited to, carotenoids (tetraterpenoids), monoterpenes, saponins, lipids, triterpenoid, and the like.

Betalains include, but are not limited to, betacyanins, and betaxanthins, and the like.

Organosulfides include, but are not limited to, dithiolthiones, thiosulfonates and the like. Indoles glucosinolates include, but are not limited to, indole-3-carbinol, sulforaphane, 3,3′ diindolylmethane or DIM, sinigrin, allicin, alliin, allyl isothiocyanate, piperine, syn-propanethial-S-oxide, and the like. Protein inhibitors include, but are not limited to, protease inhibitors. The other organic acids include, but are not limited to, oxalic acid, phytic acid, tartaric acid, anacardic acid, and the like.

Of the phytochemicals identified above, in an embodiment the dietary supplement is a composition that may comprise L-arabinose and one or more select compounds, such as, for example, polyphenol, flavonoids, inulin, phlorizin, coriolus versicolor, gymnema sylvestre extract, and terpenes.

It will be appreciated that arabinose is a monosaccharide (molecular formula: C₅H₁₀O₅) molecule containing five carbon atoms that inhibits the breaking down of sucrose into fructose and glucose within the intestines of mammals. Arabinose is a naturally occurring sugar found in fruits and other plants, such as corn. L-arabinose is found in nature as a component of biopolymers, such as pectin and hemicellulose. L-arabinose can be harvested from a variety of sources including the stalks of corn and corn cobs.

An embodiment of the disclosure is a composition that may comprise L-arabinose combined with one or more select compounds, such as for example polyphenol, flavonoids, inulin, phlorizin, coriolus versicolor, gymnema sylvestre extract, and terpenes to produce a nutritional supplement that inhibits the breakdown of sucrose so that the sucrose simply passes through the body naturally without being absorbed into the blood stream or used to build fat cells. Advantageously, compositions in accordance with the disclosure not only inhibit sucrose breakdown by a ratio of up to 20:1 (with approximately 5 times of affinity to sucrase than sucrose, one part of L-arabinose inhibits up to 20 parts of sucrose compounds to be broken down into fructose and glucose), but also provide other health benefits.

For example, the phytochemical inulin serves as a prebiotic for the probiotics in the digestive system of the mammal. A prebiotic is a non-digestible sugar or dietary fiber serving as food for probiotics. Advantageously, combining inulin with L-arabinose and sucrose, which has not been broken down into fructose and glucose, improves the operation of the digestive system. The L-arabinose inhibits the breakdown of sucrose into fructose and glucose and is also non-digestible by the mammal's digestive system. Similarly, inulin is non-digestible by the mammal's digestive system. The L-arabinose and inulin are non-digestible carbohydrates, and may also be referred to as dietary fiber.

Because L-arabinose and inulin are non-digestible, they travel along the digestive system and into the colon. Once in the colon, the non-digestibles survive a natural fermentation process in the colon. It should be noted that the non-digestibles include L-arabinose, inulin, and the sucrose, which the L-arabinose prevented from being broken down into fructose and glucose. Consequently, the colon has an increased supply of dietary fiber or non-digestible carbohydrates. These non-digestible carbohydrates (sucrose, L-arabinose, and inulin) operate as prebiotics and provide an increased food supply for probiotics, which are bacteria and other microorganisms naturally occurring in the colon. Consequently, the probiotics multiply and increase in their activity, which results in other health benefits that are obtained from an increased presence of probiotics in the colon, including a softening of the stool in the colon. Among other health benefits of probiotics, probiotics help to break-up any potential build-up of solidifying waste in the colon. Built-up waste in the colon can cause toxins to be produced, which may be harmful to the body. Advantageously, the probiotics facilitate natural discharge of this waste.

In an embodiment of the disclosure, L-arabinose may be combined with extracts from fungi, such as from the genus Ganoderma, including coriolus versicolor, also known as “the cloud mushroom,” to provide protection of digestive system from infection and inflammation caused by unprocessed sucrose. In an embodiment of the disclosure, L-arabinose may be combined with polysaccharides extracted from the cloud mushroom (Chinese name “Yun Zhi”). The cloud mushroom extract is available from Nantong Sihai Plant Extracts Co., Ltd. in the Jiangsu province near Shanghai and more information on the extract can be found here: http://ntsihai.en.made-in-china.com/product/xHEQDJAlKnaL/China-Cloud-Mushroom-Extract.html (last visited Oct. 25, 2012). Those of skill in the art will recognize that in various other embodiments, the L-arabinose may be combined with other fungi, such as other extracts from the Ganoderma genus, including the Ganoderma lucidum type species (also known as the “Reshi Mushroom”).

In an embodiment, a composition in accordance with the disclosure is a combination that may comprise L-arabinose and bioactive constituents of the herb gymnema sylvestre. Gymnema sylvestre is an herb native to the tropical forests of southern and central India where it has been used as a natural treatment for diabetes for nearly two millennia. See http://en.wikipedia.org/wiki/Gymnema_sylvestre (last visited Oct. 25, 2012). Gymnema sylvestre is an herb having bioactive constituents that are a group of oleanane-type triterpenoid saponins known as gymnemic acids. At least one study has found that gymnema sylvestre extracts facilitate insulin secretion in humans and other mammals. Those of skill in the art will recognize that in various other embodiments a composition in accordance with the disclosure may be a combination of L-arabinose and various forms and types of gymnemic acids.

In an embodiment, a composition in accordance with the disclosure is a combination that may comprise L-arabinose and phlorizin. Phlorizin is a type of flavonoid. See http://en.wikipedia.org/wiki/Phlorizin (last visited Oct. 25, 2012). Phlorizin is a phytochemical that can be extracted from the bark of fruit trees such as apple trees, cherry trees, and the like. Advantageously, phlorizin also serves in the body of the mammal to provide a secondary block of sugar. Blood in the mammal body is filtered by the kidneys. Sodium-dependent glucose cotransporters (SGLT) (SGLT-1 in the small intestine and SGLT-2 in proximal tubule of nephrons in the kidney) replace sugar removed from the blood due to filtration done by the kidneys. Without the SGLT-1 and SGLT-2 cotransporters, sugar in the blood is removed by the kidneys and expelled through the bladder. Phlorizin serves to inhibit the recycling of sugar by the SGLT-1 cotransporter so that less sugar is recycled. This further reduces the sugar level in the blood and removes the sugar as an energy source that the body may store in fat cells. Those of skill in the art will recognize that in various other embodiments a composition in accordance with the disclosure may be a combination of L-arabinose and various forms and types of flavonoids.

In an embodiment, a composition in accordance with the disclosure is a combination that may comprise L-arabinose and bioactive constituents of ginkgo biloba. The bioactive constituents may be extracted from the ginkgo biloba, for example from the gingko leaves. Ginkgo biloba may also be known by the names of “yin xing” or “icho ginnan” and may be spelled gingko. See http://en.wikipedia.org/wiki/Ginkgo_biloba (last visited Oct. 25, 2012). Ginkgo biloba is a type of tree often found in Asia. The ginkgo biloba extract is taken from Ginkgo leaves which contain flavonoid glycosides and terpenoids (ginkgolides, bilobalides). Advantageously, ginkgo biloba extract has proven effective in treating dementia in certain studies. Those of skill in the art will recognize that in various other embodiments a composition in accordance with the disclosure may be a combination of L-arabinose and various forms and types of flavonoids.

One potential benefit of the compositions in accordance with the disclosure is a shift in the body's reliance on the quick readily available energy source of fructose and glucose to other non-sucrose macronutrients. By ingesting a composition in accordance with the disclosure, the body absorbs significantly less sucrose (one part of the L-arabinose in the composition in accordance with the disclosure inhibits up to 20 parts sucrose), the sucrose may then later serve as a food supply to probiotics in the digestive tract or pass through the digestive system. Consequently, the body shifts from reliance on the ready energy source of fructose and glucose to other macronutrients such as fatty acids (fat), amino acids (protein), and other carbohydrates other than sucrose.

Macronutrients include carbohydrates, proteins, and fats. Macronutrients are nutrients that the mammal body needs in large quantities. Carbohydrates include saccharides, such as sucrose, but also include sugars other than glucose. These macronutrients, proteins, fats, and carbohydrates other than sucrose, are referred to herein as “non-sucrose macronutrients.” It is believed that the body shifts from relying on sucrose as an energy source to the non-sucrose macronutrients because the body still has a minimum or average number of calories that are needed each day (typically the average number of calories needed each day by humans is about 2,000 calories) to sustain itself. The body makes up for the deficit in calories caused by the sucrose that is not broken down by the sucrase of the body.

Another potential advantage of compositions in accordance with the disclosure is improved digestive system function in the mammal. Because the L-arabinose inhibits the breakdown of sucrose in the system, the sucrose that is not broken down into fructose and glucose remains as a disaccharide throughout the digestive tract. The increased level of sucrose in the digestive tract provides an additional food source, i.e., a prebiotic, for use by probiotics and encourages increased numbers of probiotics. Probiotics are live microorganisms, including bacteria, which benefit the digestive tract. Examples of a probiotic include, but are not limited to, bifidobacterium, clostridium butyricum, and the like. The increased food source (the unbroken down sucrose) leads to increased numbers of probiotics in the digestive system. The increased numbers of probiotics improve the health of the digestive system by reducing constipation and softening the stool to aid in proper function of the digestive system.

Advantageously, this shift to other non-sucrose macronutrients provides health benefits. Ingestion of compositions disclosed in the embodiments of the disclosure may result in reduced levels of glucose in the blood. Consequently, the body turns to fat cells to provide glucose to produce energy when needed. The shifting of the body to burning more fat aides in weight control.

The dietary supplement of the disclosure can be formulated from using any commercially acceptable form of L-arabinose and one or more of the select compounds. The dietary supplement of the disclosure may include one or more binders, fillers, flavorings, buffers, gels, excipients, carriers, or other compounds that facilitate the formulation or administration of the dietary supplement. The composition in accordance with the disclosure can be formulated into tablets, granules, powders, gels, or liquids (a tablet as used herein refers to any form of a solid oral dosage, including but not limited to tablets, caplets, capsules, powders, and the like.). Preferably, the composition is ingested into the body as a dietary supplement. As the composition passes through the intestines, the L-arabinose inhibits the breakdown of sucrose in the system and the other constituents of the composition provide additional health benefits, including those described above.

In an embodiment, the dietary supplement may be administered in three dosages of two pills each dose per day; however the supplement can be administered in other dosages and forms as desired. Alternatively, or in addition, the dosage level may vary based on the amount of sucrose a person or mammal ingests. By way of example, an average meal consumed by a human has approximately 15 grams of sucrose. In an embodiment, each pill, or tablet, inhibits the breakdown of up to 10 grams of sucrose. Consequently, for an average meal a dosage of 2 pills taken with the meal is sufficient to block the break down of a high percentage of the sucrose consumed in the meal. The effects of each dosage last for about 2 hours after being taken.

The disclosure provides a dietary supplement composition by mixing appropriate unit doses of L-arabinose and one or more select compounds described herein to obtain a composition suitable for reducing blood sugar levels in humans or animal subjects in addition to other health benefits.

Active new dietary supplement compositions of the disclosure may be produced by the procedures described herein or variations thereof, which will be apparent to one of skill in the art.

A further aspect of the disclosure is a pharmaceutical formulation comprising a composition as described above in a pharmaceutically acceptable carrier (e.g. an aqueous or a non aqueous carrier).

A still further aspect of the disclosure is a method of reducing blood sugar levels in a human or animal subject and/or of improving digestive health in the human or animal subject by administering a treatment effective amount (e.g. an amount effective to treat, reduce, or mitigate the break down of sucrose into glucose and fructose) of a composition as described above.

Embodiments of different compositions and methods of making the compositions of the disclosure are illustrated in the following examples.

### EXAMPLE 1

A single tablet is prepared comprising both L-arabinose and a selected phytochemical compound selected from the group consisting of phlorizin, coriolus versicolor, and gymnema sylvestre extract.

The tablet includes L-arabinose in a range of about 500 milligrams to about 1000 milligrams and coriolus versicolor extract in a range of about 50 milligrams to about 250 milligrams. In certain embodiments, the tablet may include between a range of about twenty parts to about one part, or about fifteen parts to about eight parts, or about ten parts L-arabinose for every one part coriolus versicolor extract.

Next a digestible binder such as maltodextrin or konjac mannan may be added in an effective amount to hold the L-arabinose and coriolus versicolor extract together in tablet form. Optionally, a flavoring food additive may be added such as anise, peppermint, spearmint, vanilla, or the like. The composition may then be agitated. Next, the mixture is placed in a tablet press machine such as the TDP series single punch tablet press available from Shanghai Minsheng Pharmaceutical Machinery Co., Limited of Shanghai China. The pressed tablets may then be packaged for the consumer. Of course in other embodiments, the dry mixed composition may be granulated and placed in ingestible capsules or other ingestible forms.

In this example, one tablet may provide approximately 500 milligrams (and up to about 1,000 milligrams) of L-arabinose, which inhibits catabolism of as much as 10 grams of the sucrose consumed by the taker of the dietary supplement. In addition, the selected compound, coriolus versicolor extract, provides the numerous health benefits described above.

### EXAMPLE 2

A single tablet is prepared comprising both L-arabinose and a selected phytochemical compound selected from the group consisting of phlorizin, coriolus versicolor, and gymnema sylvestre extract.

The composition may include L-arabinose within a range of about 100 milligrams to about 250 milligrams and phlorizin in a range of about 100 milligrams to about 200 milligrams. In certain embodiments, the composition includes about one part L-arabinose for every one part phlorizin. After adding the ingredients, the composition may be agitated. Next, the mixture is placed in a digestible capsule (i.e gelatin capsules) or other ingestible form.

In this example, one capsule may provide approximately 250 milligrams of L-arabinose, which inhibits catabolism of as much as 5 grams of the sucrose consumed by the taker of the dietary supplement. In addition, the selected compound, phlorizin, provides the numerous health benefits described above.

### EXAMPLE 3

A single tablet is prepared comprising both L-arabinose and a selected phytochemical compound selected from the group consisting of phlorizin, coriolus versicolor, and gymnema sylvestre extract.

The composition may include L-arabinose in a range of about 150 milligrams to about 600 milligrams, or about 300 milligrams and gymnema sylvestre extract in a range of about 50 milligrams to about 200 milligrams, or about 100 milligrams. In certain embodiments, the composition includes about three parts L-arabinose for every one part gymnema sylvestre extract. After adding the ingredients, the composition may be agitated. Next, the mixture is placed in a digestible capsule (i.e gelatin capsules) or other ingestible form.

In this example, one capsule provides approximately 300 milligrams of L-arabinose which inhibits catabolism of as much as 6 grams of the sucrose consumed by the taker of the dietary supplement. In addition, the selected compound, gymnema sylvestre extract, provides the numerous health benefits described above.

It will be appreciated that all numbers, parts, percentages, and the like that fall within or between the ranges disclosed herein are intended to fall within the scope of the disclosure as if each number, part, percentage and the like was individually disclosed. For example, in a composition that may comprise a biologically effective composition comprising a range of about twenty parts to about one part of L-arabinose and one part of one or more of the select compounds, L-arabinose may be present in the composition in any amount that falls within the range of one part and twenty parts, whether the exact number is exactly specified or not. Thus, by way of example, three and three-fifths parts of L-arabinose, while not individually disclosed, should be viewed as part of the disclosure since that number falls directly between one part and twenty parts, which was specifically disclosed.

Those of skill in the art will recognize different variations and modifications that can be made to compositions and method in accordance with the disclosure without departing from the scope or spirit of the disclosure as defined by the claims that follow.

In the foregoing Detailed Description, various features of the disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description by this reference, with each claim standing on its own as a separate embodiment of the disclosure.

It is to be understood that the above-described arrangements are only illustrative of the application of the principles of the disclosure. Numerous modifications and alternative arrangements may be devised by those skilled in the art without departing from the spirit and scope of the disclosure and the appended claims are intended to cover such modifications and arrangements. Thus, while the disclosure has been described above with particularity and detail, it will be apparent to those of ordinary skill in the art that numerous modifications, including, but not limited to, variations in amount, ingredients, form, function and manner of operation, assembly and use may be made without departing from the principles and concepts set forth herein.

What is claimed is:

1. An all natural dietary supplement composition comprising:
an effective amount of L-arabinose; and
an effective amount of a phytochemical compound;
wherein L-arabinose is present in an amount that is greater than about fifty percent by weight of the composition.

2. A dietary supplement composition comprising:
an effective amount of L-arabinose to mitigate catabolism of sucrose into glucose and fructose thereby increasing levels of sucrose in a user's digestive tract, such that the sucrose is usable as a prebiotic for use by probiotics within the user's digestive system; and
an effective amount of a phytochemical compound.

3. The dietary supplement composition of claim 2, wherein the phytochemical compound is a fungus.

4. The dietary supplement composition of claim 3, wherein the fungus is coriolus versicolor saccharides, wherein L-arabinose is present in a range of about 500 milligrams to about 1000 milligrams and coriolus versicolor is present in a range of about 50 milligrams to about 250 milligrams.

5. The dietary supplement composition of claim 2, wherein L-arabinose is present in the composition in an amount that is greater than about fifty percent by weight.

6. The dietary supplement composition of claim 5, wherein L-arabinose is present in the composition in an amount that is between a range of about eighty percent to about ninety-five percent by weight.

7. The dietary supplement composition of claim 2, wherein the phytochemical compound is selected from the group comprising one or more of phlorizin, coriolus versicolor, fomes yucatanensis, white kidney bean and gymnema sylvestre extract.

8. The dietary supplement composition of claim 2, wherein the phytochemical compound is selected from the group comprising one or more of a terpene (isoprenoid), a polyphenolic compound, and a saponin.

9. The dietary supplement composition of claim 2, wherein the phytochemical compound composition is phlorizin, and wherein L-arabinose is present in the composition within a range of about 100 milligrams to about 250 milligrams and phlorizin is present in a range of about 100 milligrams to about 200 milligrams.

10. The dietary supplement composition of claim 2, wherein the phytochemical compound is gymnema sylvestre extract, wherein L-arabinose is present in the composition within a range of about 150 milligrams to about 600 milligrams and gymnema sylvestre extract is present within a range of about 50 milligrams to about 200 milligrams.

11. The dietary supplement composition of claim 2, wherein the phytochemical compound is ginkgo biloba.

12. The dietary supplement composition of claim 2, wherein the phytochemical compound is a betalain.

13. The dietary supplement composition of claim 2, wherein the phytochemical compound is an inulin.

14. The dietary supplement composition of claim 2, wherein the phytochemical compound is an organosulfide.

15. The dietary supplement composition of claim 2, wherein the phytochemical compound is an indole.

16. The dietary supplement composition of claim 2, wherein the phytochemical compound is a glucosinolate.

17. The dietary supplement composition of claim 2, wherein the phytochemical compound is a protein inhibitor.

18. The dietary supplement composition of claim 2, wherein the phytochemical compound is an organic acid.

19. The dietary supplement composition of claim 2, wherein the composition comprises two or more phytochemical compounds selected from the group comprising phlorizin, coriolus versicolor, fomes yucatanensis, white kidney bean and gymnema sylvestre extract.

20. The dietary supplement composition of claim 2, wherein the phytochemical compound comprises one or more compounds selected from the group consisting of phenolic compounds, terpenes (isoprenoids), betalains, organosulfides, indoles, glucosinolates, protein inhibitors, and other organic acids.

21. The dietary supplement composition of claim 2, wherein the phytochemical compound is present in the composition in an amount that is less than about fifty percent by weight.

22. The dietary supplement composition of claim 21, wherein the phytochemical compound is present in the composition in an amount that is within a range of about twenty percent to about five percent by weight.

23. A method of managing blood sugar levels of a subject comprising the step of administering the composition of claim 2 to a subject, wherein the composition delivers an effective amount of L-arabinose and one or more selected phytochemical compounds to the subject.
